(19) United States
(12) Patent Application Publication
(10) Pub. No.: US 2018/0236205 A1
(43) Pub. Date: Aug. 23, 2018

(54) LOADING TOOLS FOR USE WITH MEDICAL DEVICES

(71) Applicant: BOSTON SCIENTIFIC SCIMED, INC., Maple Grove, MN (US)

(72) Inventors: DANIEL LEE KRAUTKREMER, PLYMOUTH, MN (US); DANIEL J. GREGORICH, PLYMOUTH, MN (US); JOHNATHAN J. BRAUN, BROOKLYN PARK, MN (US); KATHERINE STRYKER BRODEEN ROUTH, COON RAPIDS, MN (US); GARY JOHN PEDERSON, JR., ALBERTVILLE, MN (US); JAYDEEP Y. KOKATE, PLYMOUTH, MN (US); JORDAN POLLACK, MINNEAPOLIS, MN (US); TIMOTHY A. OSTROOT, COKATO, MN (US); DAVID RAAB, ROSEVILLE, MN (US); JAMES M. ANDERSON, CORCORAN, MN (US)

(73) Assignee: BOSTON SCIENTIFIC SCIMED, INC., MAPLE GROVE, MN (US)

(21) Appl. No.: 15/903,022

(22) Filed: Feb. 22, 2018

Related U.S. Application Data
(60) Provisional application No. 62/462,682, filed on Feb. 23, 2017.

(51) Int. Cl. A61M 25/01; A61M 25/06

(52) U.S. Cl. CPC A61M 25/0172; A61M 25/10; A61M 25/0668; A61M 25/0111

(57) ABSTRACT

Loading tools for use with medical devices are disclosed. An example loading tool may include a tubular sleeve having a proximal end region, a distal end region, and a lumen. The distal end region may be designed to be engaged with a resilient seal member of a hemostasis valve so that a medical device disposed within the lumen can be passed through the resilient seal member. The tubular sleeve may include a tube wall. At least a portion of the tube wall may overlap to define an overlapping region.

### CROSS-REFERENCE TO RELATED APPLICATIONS

This application claims the benefit of priority under 35 U.S.C. § 119 to U.S. Provisional Application Ser. No. 62/462,682, filed Feb. 23, 2017, the entirety of which is incorporated herein by reference.

### TECHNICAL FIELD

The present disclosure pertains to medical devices, and methods for manufacturing medical devices. More particularly, the present disclosure pertains to loading tools for use with medical devices.

### BACKGROUND

A wide variety of intracorporeal medical devices have been developed for medical use, for example, intravascular use. Some of these devices include guidewires, catheters, and the like. These devices are manufactured by any one of a variety of different manufacturing methods and may be used according to any one of a variety of methods. Of the known medical devices and methods, each has certain advantages and disadvantages. There is an ongoing need to provide alternative medical devices as well as alternative methods for manufacturing and using medical devices.

### BRIEF SUMMARY

This disclosure provides design, material, manufacturing method, and use alternatives for medical devices. An example medical device includes a loading tool for use with a drug-coated expandable medical device. The loading tool comprises: a tubular sleeve having a proximal end region, a distal end region, and a lumen; wherein the distal end region is designed to be engaged with a resilient seal member of a hemostasis valve so that a medical device disposed within the lumen can be passed through the resilient seal member; wherein the tubular sleeve includes a tube wall; and wherein at least a portion of the tube wall overlaps to define an overlapping region.

Alternatively or additionally to any of the embodiments above, the tubular sleeve comprises an inner member and an outer member.

Alternatively or additionally to any of the embodiments above, the inner member has a first length, wherein the outer member has a second length, and wherein the first length is greater than the second length.

Alternatively or additionally to any of the embodiments above, the inner member has a first stiffness, wherein the outer member has a second stiffness, and wherein the first stiffness is less than the second stiffness.

Alternatively or additionally to any of the embodiments above, the overlapping region is defined by the overlapping of the inner member by the outer member.

Alternatively or additionally to any of the embodiments above, the inner member includes an axially-extending slot.

Alternatively or additionally to any of the embodiments above, the outer member includes an axially-extending slot.

Alternatively or additionally to any of the embodiments above, the outer member is movable relative to the inner member.

Alternatively or additionally to any of the embodiments above, the tubular sleeve comprises a single tubular member and wherein the overlapping region is defined where the tubular wall circumferentially overlaps with itself.

Alternatively or additionally to any of the embodiments above, the tubular sleeve comprises a balloon protector.

A loading tool is disclosed. The loading tool comprises: an adapter body designed to facilitate loading of a medical device into a hemostasis valve; wherein the adapter body includes a distal region and a proximal region; wherein the distal region is designed to engage a resilient seal member of the hemostasis valve; and wherein the proximal region is designed to engage a balloon protector disposed about an expandable balloon such that the expandable balloon can be advanced from the balloon protector and through the resilient seal member.

Alternatively or additionally to any of the embodiments above, the distal region is tapered.

Alternatively or additionally to any of the embodiments above, the adapter body has a lumen extending therethrough and wherein the lumen varies in diameter along the length of the adapter body.

Alternatively or additionally to any of the embodiments above, the adapter body has an axially-extending slit formed therein.

Alternatively or additionally to any of the embodiments above, the proximal region includes a first attachment region that is designed to releasably attach to the balloon protector.

Alternatively or additionally to any of the embodiments above, the proximal region includes a second attachment region that is designed to releasably attach to the hemostasis valve.

Alternatively or additionally to any of the embodiments above, a handle is disposed along the proximal region.

A medical device assembly is disclosed. The assembly comprises: an expandable medical device; a drug coating disposed along the expandable medical device; a loading tool disposed about the expandable medical device; and wherein the loading tool comprises: a tubular sleeve having a proximal end region, a distal end region, and a lumen, wherein the distal end region is designed to be engaged with a resilient seal member of a hemostasis valve so that a medical device disposed within the lumen can be passed through the resilient seal member, wherein the tubular sleeve includes a tube wall, and wherein at least a portion of the tube wall overlaps to define an overlapping region.

Alternatively or additionally to any of the embodiments above, the expandable medical device comprises a balloon.

Alternatively or additionally to any of the embodiments above, the expandable medical device comprises a stent.

The above summary of some embodiments is not intended to describe each disclosed embodiment or every implementation of the present disclosure. The Figures, and Detailed Description, which follow, more particularly exemplify these embodiments.

### BRIEF DESCRIPTION OF THE DRAWINGS

The disclosure may be more completely understood in consideration of the following detailed description in connection with the accompanying drawings, in which:

FIG. 1 is a side view of an example hemostasis valve assembly.

FIG. 2 is an end view of an example hemostasis valve assembly.

FIG. 3 is a side view of an example medical device.

FIG. 4 is an end view of an example loading tool and an example medical device.

FIGS. 5-6 are a views of an example loading tool and an example medical device.

FIG. 7 is a plan view of an example loading tool being used with an example medical device and with a hemostasis valve assembly.

FIG. 8 is a cross-sectional view of an example loading tool.

FIG. 9 is a cross-sectional view of an example loading tool.

FIG. 10 is a side view of an example loading tool and an example medical device.

FIG. 11 is a side view of an example loading tool.

FIG. 12 is a side view of an example loading tool and an example hemostasis valve.

FIGS. 13-14 illustrate an example loading tool and the use of the example loading tool.

While the disclosure is amenable to various modifications and alternative forms, specifics thereof have been shown by way of example in the drawings and will be described in detail. It should be understood, however, that the intention is not to limit the invention to the particular embodiments described. On the contrary, the intention is to cover all modifications, equivalents, and alternatives falling within the spirit and scope of the disclosure.

### DETAILED DESCRIPTION

For the following defined terms, these definitions shall be applied, unless a different definition is given in the claims or elsewhere in this specification.

All numeric values are herein assumed to be modified by the term “about”, whether or not explicitly indicated. The term “about” generally refers to a range of numbers that one of skill in the art would consider equivalent to the recited value (e.g., having the same function or result). In many instances, the terms “about” may include numbers that are rounded to the nearest significant figure.

The recitation of numerical ranges by endpoints includes all numbers within that range (e.g. 1 to 5 includes 1, 1.5, 2, 2.75, 3, 3.80, 4, and 5).

As used in this specification and the appended claims, the singular forms “a”, “an”, and “the” include plural referents unless the content clearly dictates otherwise. As used in this specification and the appended claims, the term “or” is generally employed in its sense including “and/or” unless the content clearly dictates otherwise.

It is noted that references in the specification to “an embodiment”, “some embodiments”, “other embodiments”, etc., indicate that the embodiment described may include one or more particular features, structures, and/or characteristics. However, such recitations do not necessarily mean that all embodiments include the particular features, structures, and/or characteristics. Additionally, when particular features, structures, and/or characteristics are described in connection with one embodiment, it should be understood that such features, structures, and/or characteristics may also be used connection with other embodiments whether or not explicitly described unless clearly stated to the contrary.

The following detailed description should be read with reference to the drawings in which similar elements in different drawings are numbered the same. The drawings, which are not necessarily to scale, depict illustrative embodiments and are not intended to limit the scope of the invention.

A number of medical procedures, for example intravascular procedures, utilize medical devices within body lumens. For example, some intravascular procedures include the placement of a guidewire, guide catheter, balloon catheter, stent delivery system, interventional device, or the like in a blood vessel. Because fluid under pressure (e.g., blood) is present within the blood vessel, the fluid could travel along or through the medical device and escape or leak from the patient. In some instances, it may be desirable to dispose a hemostasis valve or hemostasis valve assembly at the proximal end of a medical device to reduce or otherwise limit the leaking of fluids/blood from the proximal end of the device. This may include a hemostasis valve that can be secured to a guide catheter, a hemostasis valve that can be secured to an introducer or other medical device, a hemostasis valve on the proximal end of an introducer, or the like.

A variety of different hemostasis valve designs may be used. At least some of these designs may include a resilient seal member with one or more slits or openings formed therein that allow a device to be passed therethrough. The opening or openings are designed to deform when the device is passed therethrough in a manner that opens up the resilient seal member sufficiently to allow the medical device to pass while also conforming to the outer surface of the medical device in order to effect a sufficient seal. When passing a medical device through the resilient seal member, it can be appreciated that the resilient seal may exert a force onto the medical device. In many cases, the forces exerted on the medical device may have little or no impact on the medical device. However, in some instances, if the hemostasis valve includes a resilient seal member with a single, relatively small opening, the force exerted on the medical device could be sufficient to remove or partially remove a coating (e.g., a therapeutic coating disposed along an expandable medical device such as a balloon or stent) from the medical device. It may be desirable to limit forces between the resilient seal member and the medical device. By limiting these forces, the amount of the coating and/or drug that may be removed from the medical device during the passing of the medical device through the resilient seal member may be reduced or otherwise eliminated. In addition, by reducing the forces, it is also less likely that the medical device may cause the resilient seal to be torn or otherwise deformed in a way that cannot be recovered when the medical device is passed through the resilient seal member.

Disclosed herein are loading tools that may be used with medical devices. The loading tools are designed so that a medical device can be passed through a number of different hemostasis valves. For example, the loading tools disclosed herein are designed so that a medical device can be passed through a resilient seal member (e.g., including a resilient seal member with a single, relatively small opening) in a manner that reduces the forces applied by the resilient seal member on the medical device. This may reduce or eliminate the likelihood that a coating/drug may be removed from the medical device, may reduce or eliminate the likelihood that a resilient seal may be torn or otherwise deformed in a way that cannot be recovered, and/or provide additional benefits. In addition, the loading tools disclosed herein also allows a medical device to be loaded into a hemostasis valve while the medical device, particularly a portion of the medical device that includes a drug coating (e.g., a drug coated stent, a drug coated balloon, etc.), is covered or otherwise shielded from contact by the clinician.

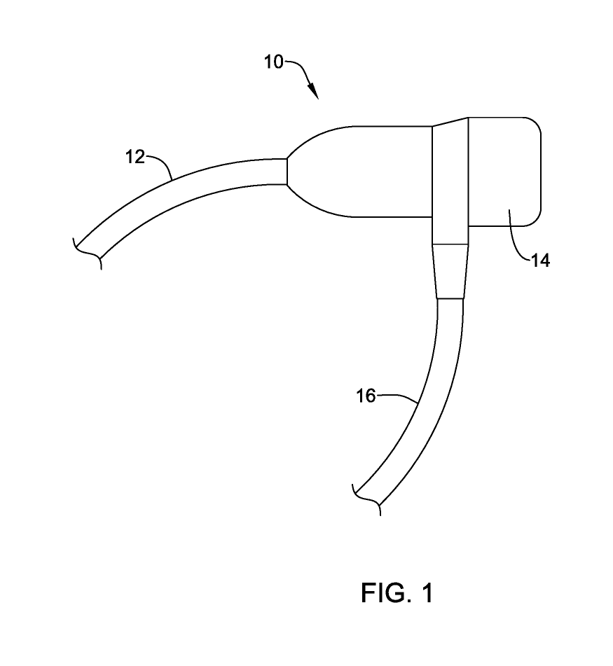

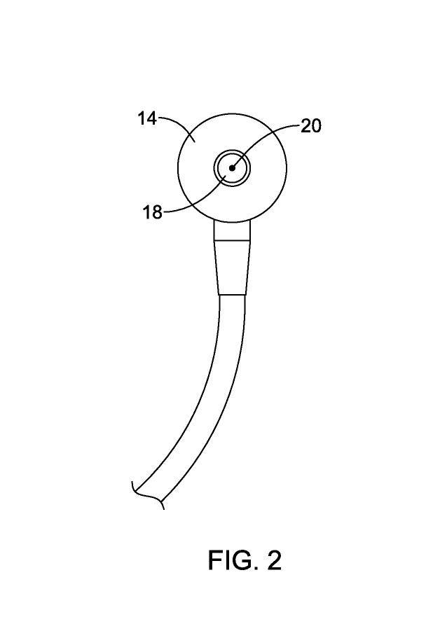

FIG. 1 illustrates an example hemostasis valve assembly 10. In this example, the hemostasis valve assembly 10 includes an introducer sheath 12 with a hemostasis valve 14 disposed at a proximal end of the introducer sheath 12. The hemostasis valve 14 may have a side port 16 that may be used to infuse fluids or the like. As shown in FIG. 2, the hemostasis valve 14 may include a resilient seal member 18 having an opening 20 formed therein. In this example, the hemostasis valve 14 includes only a single opening 20. However, other hemostasis valves 14 may have differing arrangements including slits, cross-slits, star-shaped slits, or the like. In general, the opening 20 may be designed to deform when a medical device is passed therethrough in a manner that allows the medical device to pass therethrough while also conforming to the outer surface of the medical device in order to effect a sufficient seal.

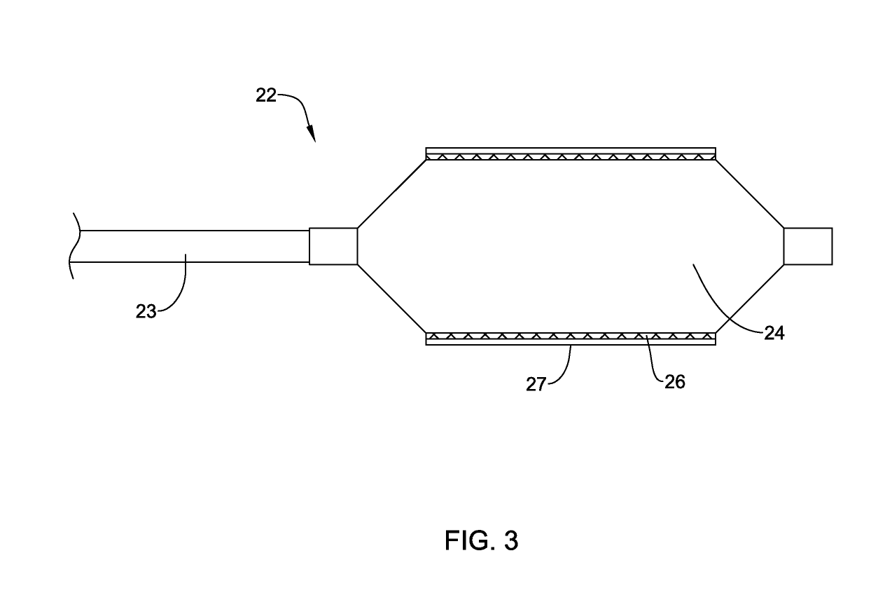

An example medical device 22 that may be passed through the resilient seal member 18 is shown in FIG. 3. The example medical device 22 may take the form of a stent delivery system including a catheter shaft 23, an expandable balloon 24 coupled to the catheter shaft 23, an expandable stent 26 coupled the balloon 24, and a coating 27 (e.g., a therapeutic or drug coating 27) coupled to the stent 26. This is just one example. Other medical devices contemplated include balloon catheters, self-expanding or other stent delivery systems, drug coated balloons, systems for delivering other implantable medical devices including prosthetic valves, catheters, or the like.

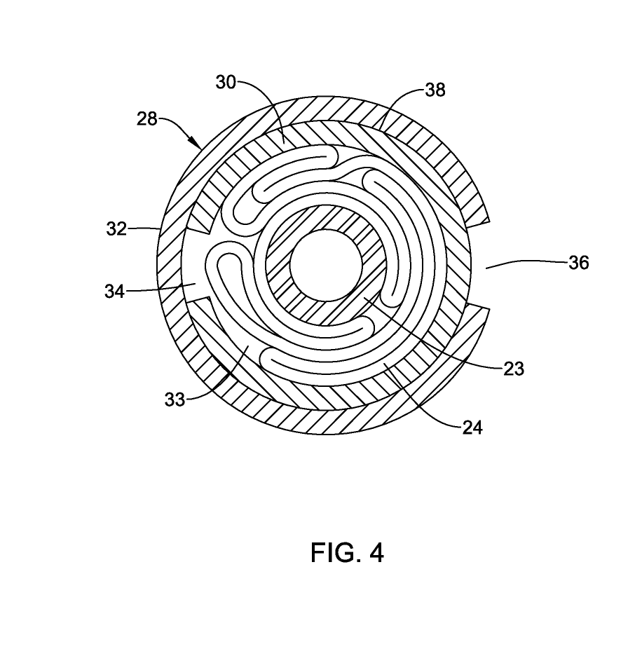

In order to efficiently navigate the medical device 22 through the resilient seal member 18 of the hemostasis valve 14, a loading tool 28 may be utilized such as the loading tool 28 depicted in FIG. 4. In general, the loading tool 28 is designed so that the medical device 22 can be passed through the resilient seal member 18 in a manner that reduces the forces applied by the resilient seal member 18 on the medical device 22. This may reduce or eliminate the likelihood that a coating/drug 27 may be removed from the medical device 22, may reduce or eliminate the likelihood that the resilient seal member 18 is torn or otherwise deformed in a way that cannot be recovered, and/or provide additional benefits. In addition, the loading tool 28 also allows the medical device 22 to be loaded into the hemostasis valve 14 while the medical device 22, for example the drug coating 27, is covered or otherwise shielded from contact by the clinician.

The loading tool 28 may include a first or inner member 30 and a second or outer member 32. A lumen 33 may be defined in the loading tool 28. In this example, the lumen 33 may correspond to the lumen of the inner member 30. The inner member 30 may have an axially-extending slot 34 formed therein. Similarly, the outer member 32 may also have an axially-extending slot 36 formed therein. The axially-extending slots 34/36 may allow both the inner member 30 and the outer member 32 to be described as “C-shaped” tubes. Thus, the loading tool 28 may be described as including two generally coaxial, C-shaped tubes. In some instances the outer member 32 may have a greater flexural rigidity or stiffness than that of the inner member 30.

The arrangement of the inner member 30 and the outer member 32 may vary. For example, FIG. 4 illustrates the axially-extending slot 34 along the inner member 30 being disposed opposite the axially-extending slot 36 along the outer member 32. Other arrangements are contemplated. In addition, the inner member 30 and the outer member 32 may be translated or otherwise moved relative to one another, rotatable relative to one another, or both. This may allow the configuration of the inner and outer members 30/32 to be varied further.

Because the inner and outer members 30/32 may be arranged coaxially with one another at least a portion of the inner member 30 and the outer member 32 overlap with one another along an overlapping region 38. The overlapping region 38 may be defined along regions where the tube wall of the inner member 30 and the tube wall of the outer member 32 overlap. The overlapping arrangement may allow the inner member 30 and the outer member 32 to be used together to reduce the profile of the loading tool 28 as well as the profile of the medical device 22 disposed therein. The profile can be further reduced, if desired, by a force onto the outer member 32 to circumferentially compress the outer member 32. When doing so, the axially-extending slot 36 may reduce in size (as well as the axially-extending slot 34 in the inner member 30), thereby reducing the outer dimension/profile of the outer member 32, reducing the outer dimension/profile of the inner member 30, and reducing the outer dimension/profile of the medical device 22.

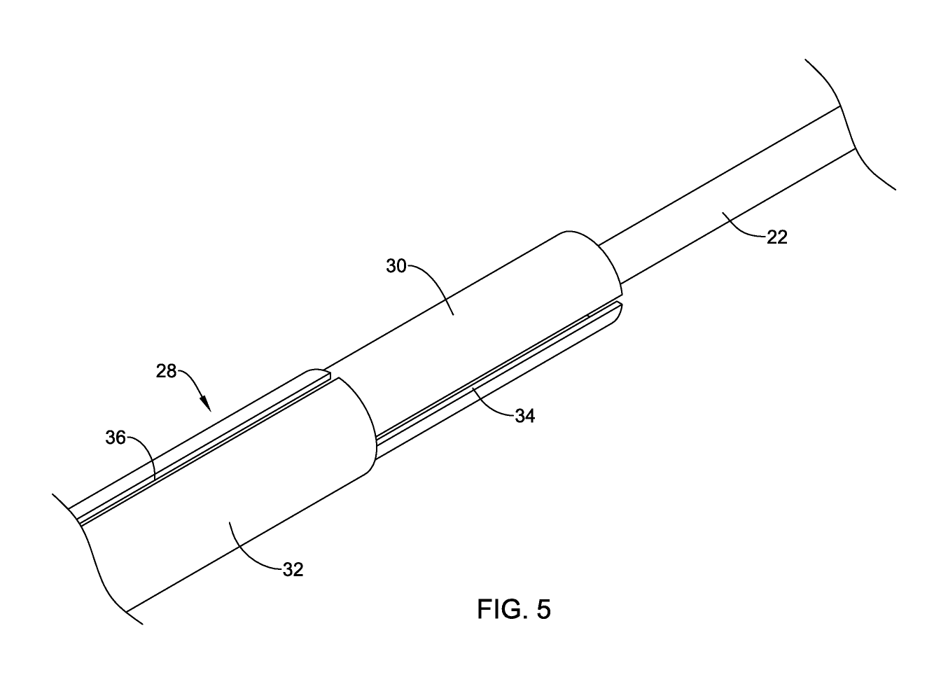

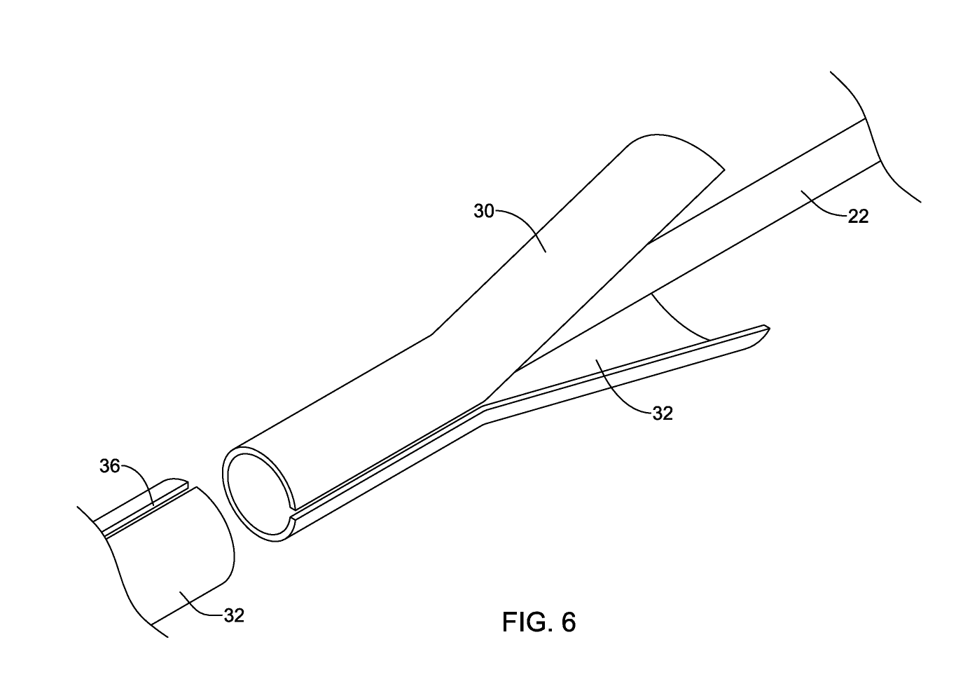

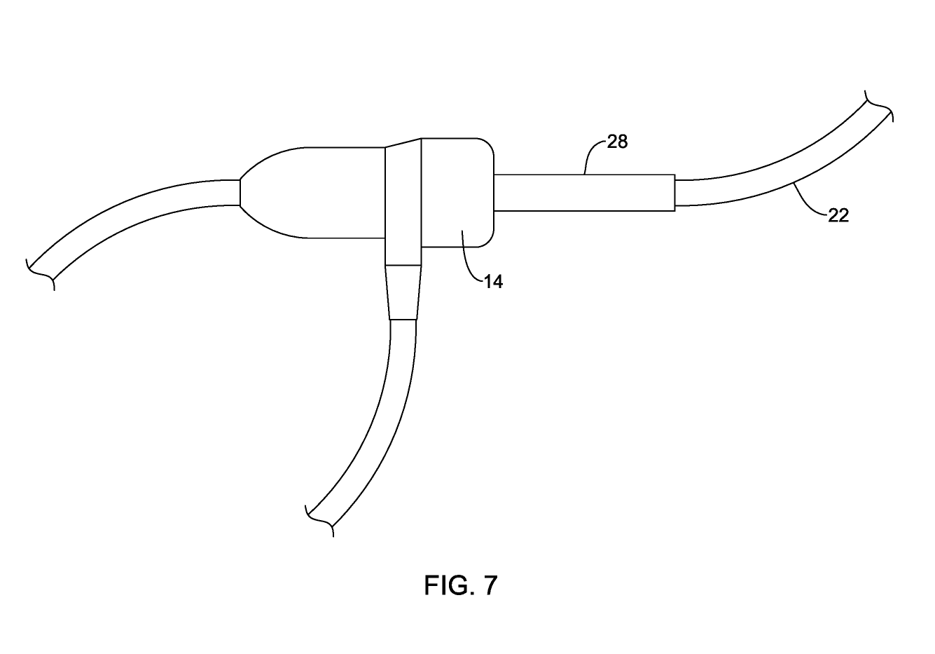

In use, the medical device 22 may be disposed at least partially within the loading tool 28. This may include removing the medical device 22 from its packaging, which may include removing a balloon protector from the balloon 24. Then the medical device 22 may be disposed within the lumen 33 of the loading tool 28 by either extending the proximal end of the medical device 22 through the distal end of the loading tool 28 (e.g., “back loading”) or extending the distal end of the medical device 22 through the proximal end of the loading tool 28. With the medical device 22 suitably arranged within the loading tool 28 (e.g., with the balloon 24, stent 26, and coating 27 disposed within the lumen 33), the outer member 32 may be slid (e.g., slid distally) off of the inner member 30 as shown in FIGS. 5-6. The inner member 30 may have a length that is greater than the length of the outer member 32. Thus, sliding the outer member 32 off of the inner member 30 may occur while a clinician is holding the inner member 30. Furthermore, in some instances, the inner member 30 can be expanded or flared outward so that a clinician may grab the medical device 22. The inner member 30 can then be inserted at least partially into the resilient seal member 18 and the medical device 22 can be urged distally through the loading tool 28, through the resilient seal member 18, into the introducer sheath 12, and into the body lumen (e.g., a blood vessel) as depicted in FIG. 7. Because the outer member 32 may be removed from the inner member 30, inserting the loading tool 28 into the resilient seal member 18 may include inserting the inner member 30 (along with the medical device 22) into the resilient seal member 18 of the hemostasis valve 14. When the medical device 22 is passed in the desired manner through the resilient seal member 18, the loading tool 28 can be proximally retracted from the resilient seal member 18. In some instances, the loading tool 28 can also be removed from catheter shaft 23. This may include cutting the loading tool 28, splitting the loading tool 28 along a perforation or line of weakness, or otherwise pulling the loading tool 28 from the catheter shaft 23. Alternatively, the loading tool 28 can be left disposed along the catheter shaft 23 and, ultimately, can be removed when the medical device 22 is removed from the body lumen.

In some of these and in other instances, the loading tool 28 may be a balloon protector. For example, the medical device 22 may be disposed within the loading tool/balloon protector 28 during packaging and a clinician can utilize the loading tool/balloon protector 28 to load the medical device 22 into the hemostasis valve 14 in a manner similar to what is described herein.

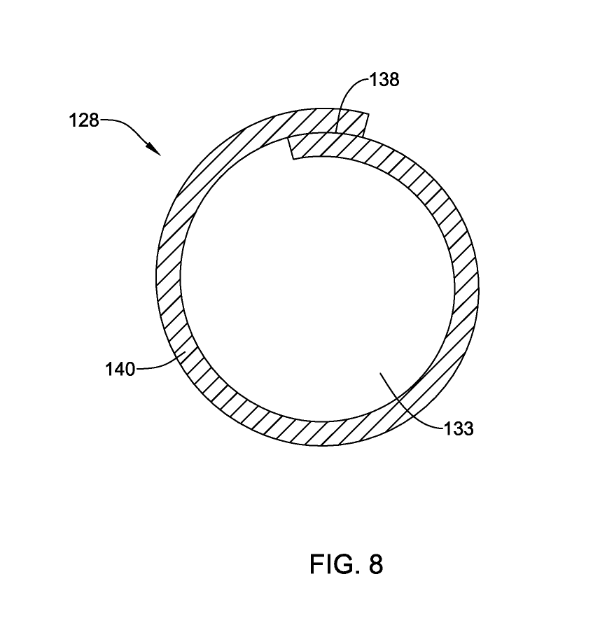

FIG. 8 illustrates another example loading tool 128 that may be similar in form and function to other loading tools disclosed herein. The loading tool 128 may include a tubular body 140 having an overlapping region 138. The overlapping region 138 may be defined where at least a portion of the tubular body circumferentially overlaps with itself. Thus, the tubular body 140 may have a shape that could be described as a single C-shaped tube (e.g., prior to the tube wall overlapping with itself), a G-shaped tube (e.g., with the tube wall overlapping with itself), or the like.

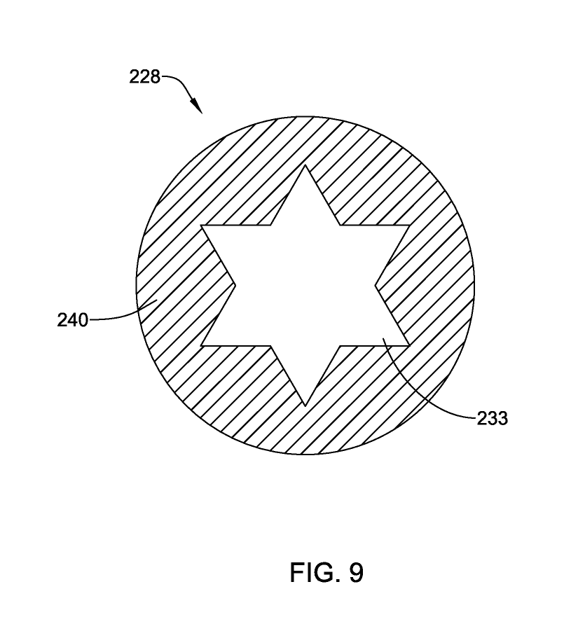

FIG. 9 illustrates another example loading tool 228 that may be similar in form and function to other loading tools disclosed herein. The loading tool 228 may include a tubular body 240 having a lumen 233 with a non-circular cross-sectional shape. In this example, the lumen 233 has a star shape. This may reduce the amount of surface area where the medical device 22 disposed in the lumen 233 may contact the inner surface of the loading tool 228. A number of differing shapes are contemplated for lumen 233. These and other non-circular cross-sectional shapes may be utilized with loading tool 228 as well as other loading tools disclosed herein.

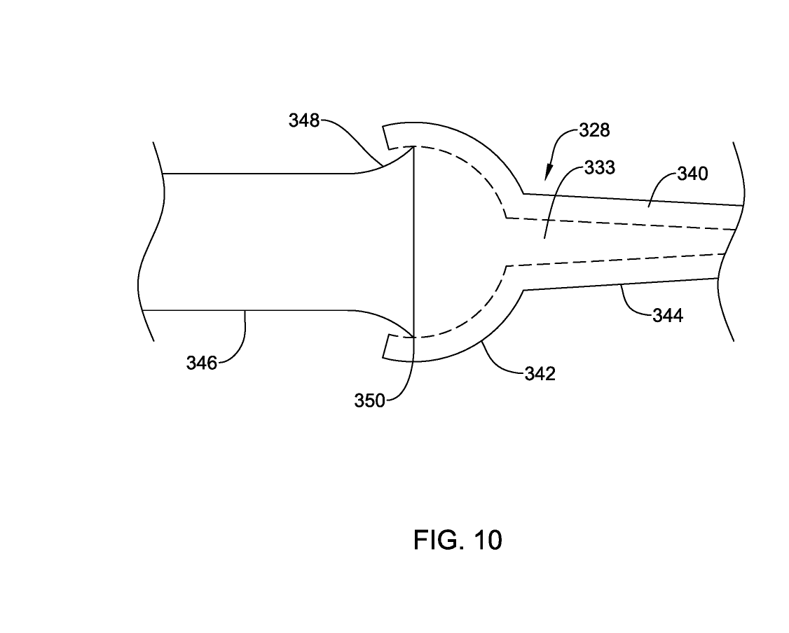

FIG. 10 illustrates another example loading tool 328 that may be similar in form and function to other loading tools disclosed herein. The loading tool 328 may include an adapter body 340 having a proximal region 342, a distal region 344, and a lumen 333. The distal region 344 may generally be designed to engage the hemostasis valve 14 (e.g., engage the resilient seal member 18). In at least some instances, the distal region 344 may be tapered. The lumen 333 may also be tapered (e.g., the inner diameter of the adapter body 340 may vary).

The proximal region 342 may generally be designed to engage a balloon protector 346 having a flared distal end 348. In at least some instance, the proximal region 342 may include a flared end with an attachment region 350 designed to engage the flared distal end 348 of the balloon protector 346. The attachment region 350 may include structural features such as snaps, grooves, slots, clips, or the like that allow the flared distal end 348 of the balloon protector 346 to clip or snap into the attachment region 350.

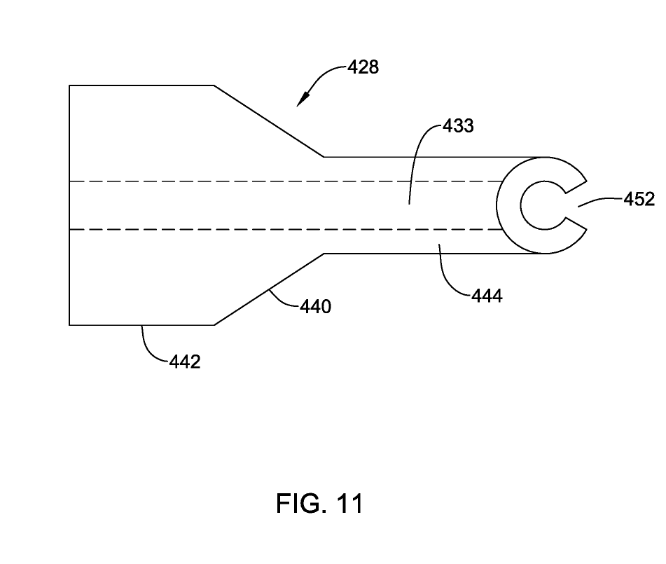

FIG. 11 illustrates another example loading tool 428 that may be similar in form and function to other loading tools disclosed herein. The loading tool 428 may include an adapter body 440 having a proximal region 442, a distal region 444, and a lumen 433. The distal region 444 may generally be designed to engage the hemostasis valve 14 (e.g., engage the resilient seal member 18). In at least some instances, the distal region 444 may be tapered. The lumen 433 may also be tapered (e.g., the inner diameter of the adapter body 440 may vary).

In some instance, the adapter body 440 may be a generally solid, circumferentially continuous structure. Alternatively, an axially-extending slit 452 may be disposed along the adapter body 440. The axially-extending slit 452 may allow the adapter body 440 to be efficiently disposed about a device such as a guidewire. In addition, the axially-extending slit 452 may allow the adapter body 440 to be removed from the medical device 22 after successfully navigating the medical device 22 (e.g., the balloon 24, the stent 26, the coating 27, etc.) through the resilient seal member 18.

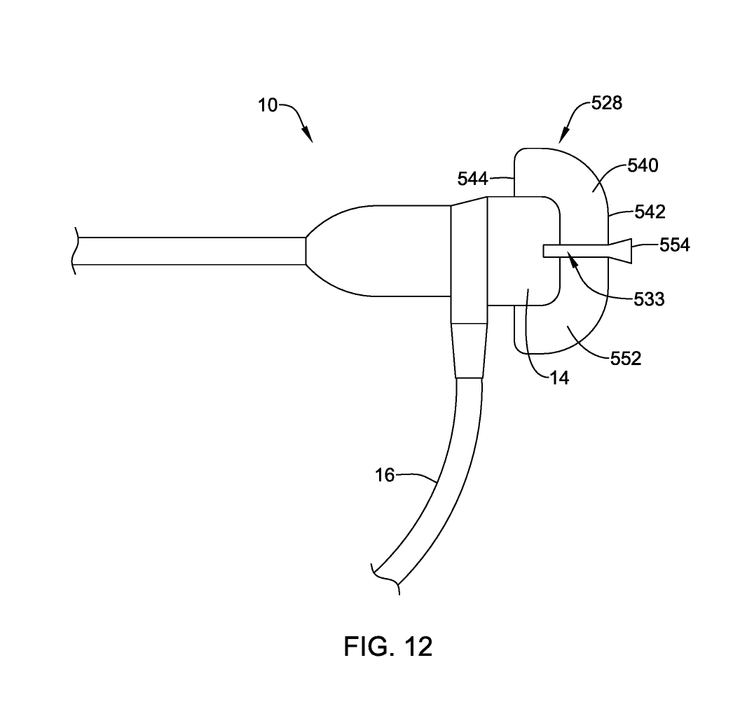

FIG. 12 illustrates another example loading tool 528 that may be similar in form and function to other loading tools disclosed herein. The loading tool 528 may include an adapter body 540 having a proximal region 542, a distal region 544, and a lumen 533. The distal region 544 may be designed to engage the resilient seal member (e.g., the resilient seal member 18). The adapter body 540 may take the form of a clip that is designed to be releasably attached to a hemostasis valve. For example, the adapter body 540 may have a first attachment region 552 that may be designed to engage and attach to, for example, a hemostasis valve (e.g., the hemostasis valve 14). The adapter body 540 may have a second attachment region 554 that may be designed to engage and attach to, for example, a balloon protector (e.g., the balloon protector 346).

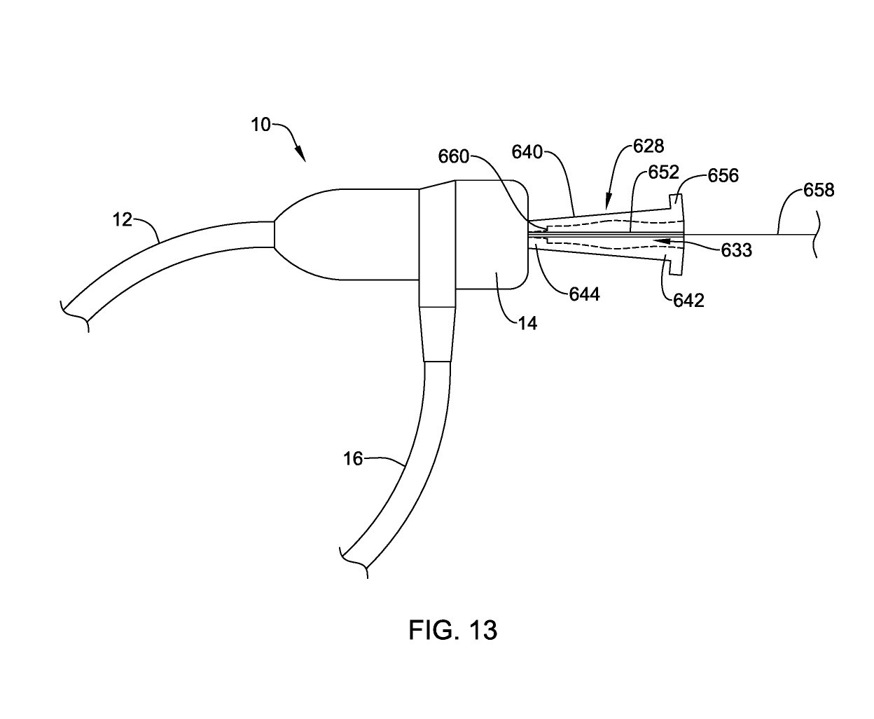

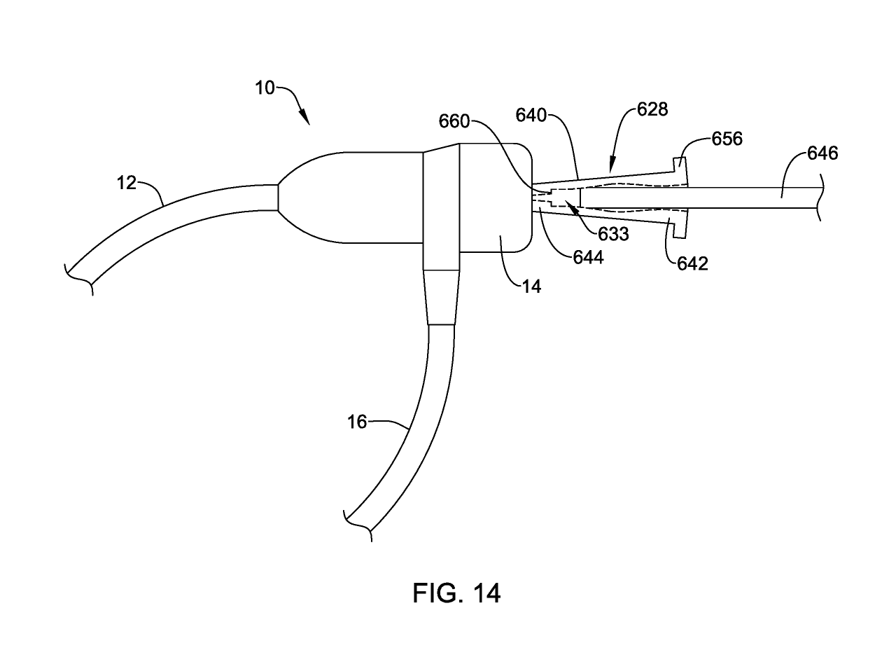

FIGS. 13-14 illustrates another example loading tool 628 that may be similar in form and function to other loading tools disclosed herein. These figures also show the use of the loading tool 628 in conjunction with the hemostasis valve assembly 10. The loading tool 628 may include an adapter body 640 having a proximal region 642, a distal region 644, and a lumen 633. The adapter body 640 may include an axially-extending slit 652. The distal region 644 may taper and be designed to extend into the resilient seal member 18 of the hemostasis valve 22. In at least some instances, the lumen 633 may also have a reduction in diameter (e.g., one or more stepped reductions 660 of the inner diameter of the distal region 644 and/or one or more tapers that vary the inner diameter). Alternatively, the lumen 633 may have a constant diameter. A handle 656 may be coupled to the proximal region 642.

In use, the loading tool 628 may be engaged with the hemostasis valve 14 as shown in FIG. 13. When doing so, the distal region 644 may be inserted into the resilient seal member 18 (not shown in FIG. 13). In some instances, the adapter body 640 may be disposed about a guidewire 658 extending from the hemostasis valve 14 (e.g., by passing the guidewire 658 through the axially-extending slit 652). A medical device 22 disposed within a balloon protector 646 may be disposed within the lumen 633 of the loading tool 628 as shown in FIG. 14. In FIG. 14, the loading tool 628 is partially cut away to shown the balloon protector 646. The balloon protector 646 (along with the medical device 22) may be advanced within the lumen 633 until reaching a point where the lumen 633 tapers to a size that stops further advancement of the balloon protector 646 or otherwise encounters the stepped reduction 660. The medical device 22 can then be advanced from the balloon protector 646, through the lumen 633, and through the resilient seal member 18.

The materials that can be used for the various components of the loading tool 28 (and/or other loading tools disclosed herein) may include those commonly associated with medical devices. For simplicity purposes, the following discussion makes reference to the loading tool 28. However, this is not intended to limit the devices and methods described herein, as the discussion may be applied to other similar loading tools or devices disclosed herein.

The loading tool 28 may be made from a metal, metal alloy, polymer (some examples of which are disclosed below), a metal-polymer composite, ceramics, combinations thereof, and the like, or other suitable material. Some examples of suitable polymers may include polytetrafluoroethylene (PTFE), ethylene tetrafluoroethylene (ETFE), fluorinated ethylene propylene (FEP), polyoxymethylene (POM, for example, DELRIN® available from DuPont), polyether block ester, polyurethane (for example, Polyurethane 85A), polypropylene (PP), polyvinylchloride (PVC), polyether-ester (for example, ARNITEL® available from DSM Engineering Plastics), ether or ester based copolymers (for example, butylene/poly(alkylene ether) phthalate and/or other polyester elastomers such as HYTREL® available from DuPont), polyamide (for example, DURETHAN® available from Bayer or CRISTAMID® available from Elf Atochem), elastomeric polyamides, block polyamide/ethers, polyether block amide (PEBA, for example available under the trade name PEBAX®), ethylene vinyl acetate copolymers (EVA), silicones, polyethylene (PE), Marlex high-density polyethylene, Marlex low-density polyethylene, linear low density polyethylene (for example REXELL®), polyester, polybutylene terephthalate (PBT), polyethylene terephthalate (PET), polytrimethylene terephthalate, polyethylene naphthalate (PEN), polyetheretherketone (PEEK), polyimide (PI), polyetherimide (PEI), polyphenylene sulfide (PPS), polyphenylene oxide (PPO), poly praraphenylene terephthalamide (for example, KEVLAR®), polysulfone, nylon, nylon-12 (such as GRILAMID® available from EMS American Grilon), perfluoro(propyl vinyl ether) (PFA), ethylene vinyl alcohol, polyolefin, polystyrene, epoxy, polyvinylidene chloride (PVdC), poly(styrene-b-isobutylene-b-styrene) (for example, SIBS and/or SIBS 50A), polycarbonates, ionomers, biocompatible polymers, other suitable materials, or mixtures, combinations, copolymers thereof, polymer/metal composites, and the like. In some embodiments the sheath can be blended with a liquid crystal polymer (LCP). For example, the mixture can contain up to about 6 percent LCP.

Some examples of suitable metals and metal alloys include stainless steel, such as 304V, 304L, and 316LV stainless steel; mild steel; nickel-titanium alloy such as linear-elastic and/or super-elastic nitinol; other nickel alloys such as nickel-chromium-molybdenum alloys (e.g., UNS: N06625 such as INCONEL® 625, UNS: N06022 such as HASTELLOY® C-22®, UNS: N10276 such as HASTELLOY® C276®, other HASTELLOY® alloys, and the like), nickel-copper alloys (e.g., UNS: N04400 such as MONEL® 400, NICKELVAC® 400, NICORROS® 400, and the like), nickel-cobalt-chromium-molybdenum alloys (e.g., UNS: R30035 such as MP35-N® and the like), nickel-molybdenum alloys (e.g., UNS: N10665 such as HASTELLOY® ALLOY B2®), other nickel-chromium alloys, other nickel-molybdenum alloys, other nickel-cobalt alloys, other nickel-iron alloys, other nickel-copper alloys, other nickel-tungsten or tungsten alloys, and the like; cobalt-chromium alloys; cobalt-chromium-molybdenum alloys (e.g., UNS: R30003 such as ELGILOY®, PHYNOX®, and the like); platinum enriched stainless steel; titanium; combinations thereof; and the like; or any other suitable material.

It should be understood that this disclosure is, in many respects, only illustrative. Changes may be made in details, particularly in matters of shape, size, and arrangement of steps without exceeding the scope of the disclosure. This may include, to the extent that it is appropriate, the use of any of the features of one example embodiment being used in other embodiments. The invention's scope is, of course, defined in the language in which the appended claims are expressed.

What is claimed is:

1. A loading tool for use with a drug-coated expandable medical device, the loading tool comprising:
a tubular sleeve having a proximal end region, a distal end region, and a lumen;
wherein the distal end region is designed to be engaged with a resilient seal member of a hemostasis valve so that a medical device disposed within the lumen can be passed through the resilient seal member;
wherein the tubular sleeve includes a tube wall; and
wherein at least a portion of the tube wall overlaps to define an overlapping region.

2. The loading tool of claim 1, wherein the tubular sleeve comprises an inner member and an outer member.

3. The loading tool of claim 2, wherein the inner member has a first length, wherein the outer member has a second length, and wherein the first length is greater than the second length.

4. The loading tool of claim 2, wherein the inner member has a first stiffness, wherein the outer member has a second stiffness, and wherein the first stiffness is less than the second stiffness.

5. The loading tool of claim 2, wherein the overlapping region is defined by the overlapping of the inner member by the outer member.

6. The loading tool of claim 2, wherein the inner member includes an axially-extending slot.

7. The loading tool of claim 6, wherein the outer member includes an axially-extending slot.

8. The loading tool of claim 7, wherein the outer member is movable relative to the inner member.

9. The loading tool of claim 1, wherein the tubular sleeve comprises a single tubular member and wherein the overlapping region is defined where the tubular wall circumferentially overlaps with itself.

10. The loading tool of claim 1, wherein the tubular sleeve comprises a balloon protector.

11. A loading tool, comprising:
an adapter body designed to facilitate loading of a medical device into a hemostasis valve;
wherein the adapter body includes a distal region and a proximal region;
wherein the distal region is designed to engage a resilient seal member of the hemostasis valve; and
wherein the proximal region is designed to engage a balloon protector disposed about an expandable balloon such that the expandable balloon can be advanced from the balloon protector and through the resilient seal member.

12. The loading tool of claim 11, wherein the distal region is tapered.

13. The loading tool of claim 11, wherein the adapter body has a lumen extending therethrough and wherein the lumen varies in diameter along the length of the adapter body.

14. The loading tool of claim 11, wherein the adapter body has an axially-extending slit formed therein.

15. The loading tool of claim 11, wherein the proximal region includes a first attachment region that is designed to releasably attach to the balloon protector.

16. The loading tool of claim 11, wherein the proximal region includes a second attachment region that is designed to releasably attach to the hemostasis valve.

17. The loading tool of claim 11, wherein a handle is disposed along the proximal region.

18. A medical device assembly, comprising:
an expandable medical device;
a drug coating disposed along the expandable medical device;
a loading tool disposed about the expandable medical device; and
wherein the loading tool comprises:
a tubular sleeve having a proximal end region, a distal end region, and a lumen,
wherein the distal end region is designed to be engaged with a resilient seal member of a hemostasis valve so that a medical device disposed within the lumen can be passed through the resilient seal member,
wherein the tubular sleeve includes a tube wall, and
wherein at least a portion of the tube wall overlaps to define an overlapping region.

19. The assembly of claim 18, wherein the expandable medical device comprises a balloon.

20. The assembly of claim 18, wherein the expandable medical device comprises a stent.
